(19) United States
(12) Patent Application Publication
(10) Pub. No.: US 2010/0018864 A1
(43) Pub. Date: Jan. 28, 2010

(54) LAB-ON-A-CHIP WITH COPLANAR MICROFLUIDIC NETWORK AND COPLANAR ELECTROSPRAY NOZZLE

(75) Inventors: Nicolas Sarrut, SEYSSINET-PARISET (FR); Olivier Constantin, GRENOBLE (FR)

Correspondence Address: Nixon Peabody LLP, P.O. Box 60610, Palo Alto, CA, 94306, US

(73) Assignee: COMMISSARIAT A L'ENERGIE ATOMIQUE, Paris (FR)

(21) Appl. No.: 12/505,395

(22) Filed: Jul. 17, 2009

(30) Foreign Application Priority Data
Jul. 24, 2008 (FR) 08 55077

(51) Int. Cl. G01N 33/48

(52) U.S. Cl. 204/601

(57) ABSTRACT

A lab-on-a-chip comprising a support plate, at least one fluidic network formed in a fluidic plate bonded onto the support plate, and a cover plate bonded onto the fluidic plate and covering the fluidic network. The fluidic network, at a first end, is connected to an inlet orifice allowing entry of a liquid to be sprayed and, at a second end, to a first end of an outlet channel for the liquid to be sprayed, formed in the fluidic plate. The fluidic plate is extended by a pointed electrospray nozzle at which the second end of the outlet channel forms the electrospray outlet of the lab-on-a-chip. The cover plate has a pointed extension forming a roof for that part of the channel located in the electrospray nozzle.

### CROSS REFERENCE TO RELATED APPLICATIONS or PRIORITY CLAIM

This application claims priority of French Patent Application No. 08 55077, filed Jul. 24, 2008.

### DESCRIPTION

1. Technical Field

The invention relates to a lab-on-a-chip comprising a coplanar microfluidic network and a coplanar electrospray nozzle. It particularly concerns the coupling of a lab-on-a-chip to a mass spectrometer

Over the last ten years, numerous pathways have been investigated for the coupling of different microfluidic devices with mass spectrometers. Optical detection methods e.g. spectrophotometry or fluorescence are not suitable for the detection of biomolecules such as proteins or peptides, this detection being of particular interest in the area of proteomics. The limits are either sensitivity or the need to prepare the sample (fluorescent tagging) which, for the identification of proteins after enzymatic digestion for example, raises a problem since the peptides obtained are theoretically not known. Mass spectrometry is therefore a frequent choice since it provides information on the nature of the analyzed samples (intensity spectrum as per the charge-to-mass ratio) with very good sensitivity (femtomole/μl), and also allows the analysis of complex mixtures of molecules. For this purpose, it is often necessary for the sample to be pre-treated prior to analysis. For example, this pre-treatment consists of separating the chemical and/or biological compounds, preceded and/or followed by concentration of the species.

For the conducting of this pre-treatment continuous with analysis within minimum time and whilst minimizing the volumes of reagents used, advantage can be taken from recent progress in the area of microfluidics. Examples thereof have already been presented, such as microfluidic enzyme digestion devices (Lian Ji Jin, “A microchip-based proteolytic digestion system driven by electroosmotic pumping”, Lab Chip, 2003, 3, 11-18), capillary electrophoresis (B. Zhang et al, “Microfabricated Devices for Capillary Electrophoresis-Electrospray Mass Spectrometry”, Anal.Chem., vol. 71, no. 15, 1999, 3259-3264) or 2D separation (J. D. Ramsey, “High-efficiency Two dimensional Separations of Protein Digests on Microfluidic Devices”, Anal. Chem., 2003, 75, 3758-3764, or N. Gottschlich et al, “Two-Dimensional Electrochromatography/Capillary Electrophoresis on a Microchip”, Anal. Chem.2001, 73, 2669-2674).

Microfluidic/mass spectrometry coupling can be based on ionization of the sample by electrospray (ElectroSpray Ionization—ESI). At atmospheric pressure and immersed in an intense electric field, the pre-treated liquid sample leaving the microfluidic chip is atomized into a gas of ions or a multitude of charged droplets able to enter the mass spectrometer (MS) for analysis. This atomization entails the deformation of the interface formed between the outgoing liquid and the surrounding gas (liquid meniscus/gas) and the <<drop>> of liquid assumes a conical shape called a <<Taylor cone>>. The volume of this cone forms a dead volume for the outgoing liquid (geometric space in which the chemical compounds may mix together), which is not desirable especially when the last step of pre-treatment precisely consists of separating the chemical compounds in the sample. This is why it is always sought to minimize the size of this cone, and inter alia this involves reducing the inner and outer dimensions of the outlet channel of the microfluidic chip.

Conventionally, during analysis by mass spectrometry, the sample is pre-treated outside the ESI device and is then manually placed (using a pipette) in a hollow needle whose tip is electrically conductive (<<PicoTip emitter>> by New Objective for example). An electric field is applied between the conductive part of the PicoTip and the MS inlet, which allows a Taylor cone to be formed at the outlet of the PicoTip and atomization of the sample. The <<pointed>> cylindrical geometry of a PicoTip is ideal for the formation of a small Taylor cone, but the limits regarding minimization of their size (conventionally outer diameter of 360 μm and inner diameter of 10 μm), the limits with respect to obtaining good reproducibility with the fabrication techniques used (pulling technique) and their fragility when used are the chief reasons prompting the search for other types of spray devices.

In the literature, when these devices are fabricated using microtechnologies such as planar silicon technologies (etching, machining, thin-layer deposit and photolithography of various materials on substrates having very large lateral dimensions compared with their thickness), mention is often made of an <<electrospray nozzle>> (Tai et al, “MEMS electrospray nozzle for mass spectroscopy”, WO-A-98/35376). Said fabrications have twofold importance.

Firstly, microtechnologies can be used to produce ESI interfaces by defining structures of pointed tip type (such as PicoTips) but smaller in size (to limit the volume of the Taylor cone), more reproducible and less fragile, which is advantageous per se (see document WO-A-00/30167).

Secondly, microtechnologies can be used to produce devices integrating a fluidic network to ensure pre-treatment of the sample and an interface of ESI type. In addition to the above-cited advantages (reduced outgoing dead volumes, reproducibility, robustness of the ESI interface), benefit is also drawn from the advantages connected with an integrated pre-treatment device (pre-treatment protocol continuous with analysis, reduction in overall analysis time, minimization of reagent volumes).

Nevertheless, said integration raises three major technical design problems:

First, the fabrication technology used for the device must be compatible with that used for a pre-treatment fluidic network (reservoirs, micro-channels, reactors) and ESI interface (tip geometry, minimal outlet dimensions . . . ) so that it is possible to fabricate the complete device on one same support or one same assembly of supports having a technological sequence common to the two integrated entities.

Secondly, it must be designed so that no additional dead volume is added to those which may exist in the pre-treatment fluidic network and in the ESI interface taken separately.

Finally, it must provide the ESI interface with a spray electrode without adding dead volume to the system. This spray electrode may be located either outside the tip structure (M. Svederberg et al, “Sheathless Electrospray from Polymer Microchips”, Anal.Chem. 2003, 75, 3934-3940) or inside the outlet channel in the vicinity of the outlet of the device. In the first case, an electric field is applied solely outside the device, in the portion of air (or other gas) located between the end of the tip and the MS inlet. In the second case, an electric field also exists inside the device, in the segment of liquid located between the electrode and the end of the tip. To implant an external electrode, it is often reported (R. B. Cole, “Electrospray ionization mass spectrometry: fundamentals, instrumentation and applications”, John Wiley & Sons: New York, 1997) that one major difficulty is to ensure its sufficient robustness. The conductive deposits made for this purpose often deteriorate too rapidly under the action of the intense electric fields.

The cover substrate can be electrically conductive.

2. State of the Prior Art

One major step forward in this area was proposed in document WO-A-2005/076 311 which discloses a microfluidic device allowing various treatments of samples and having a good interface with a mass spectrometer of ESI type, which requires:

A fabrication technology compatible with that of a pre-treatment fluidic system (reservoirs, micro-channels, reactors . . . ) and that of an outlet ESI interface (tip geometry, minimal outlet dimensions . . . ) to allow fabrication of the complete device on one same support or one same assembly of supports having a technological sequence common to the two integrated entities.

An integration design with no dead volumes.

Integration of a spray electrode inside the outlet channel in the vicinity of the outlet of the device.

This lab-on-a-chip comprises a support, at least one fluidic network, at least one fluid inlet orifice connected to the fluidic network and at least one fluid outlet orifice connected to the fluidic network. It comprises a thin layer attached onto the support and in which the fluidic network and an electrospray nozzle are fabricated. The electrospray nozzle overhangs the support and comprises a channel of which one end is connected to the fluidic network and whose other end forms said fluid outlet orifice, the channel being equipped with electric conduction means forming at least one electrode.

However, it has been ascertained that in the device described in document WO-A-2005/076 311 the flow rate of the electrospray source is limited to 0,3 μl/min. At this flow rate, overflows occur at the base of the source i.e. at the start of the outlet channel which lies in open air.

### SUMMARY OF THE INVENTION

The inventor of the present invention has investigated the possible causes of this limited flow rate and its possible remedies. The inventor has found that, by modifying the electrospray source part (or nozzle) of the different variants of the device described in document WO-A-2005/076 311, it is possible to obtain higher flow rates. This modification of the electrospray source or nozzle consists of <<closing>> the source, either by covering it with a <<roof>>, or by providing it both with a <<roof>> and a <<floor>>.

The subject of the invention is therefore a lab-on-a-chip comprising a support plate, at least one fluidic network formed in a plate called a fluidic plate bonded onto the support plate, and a plate called a cover plate bonded onto the fluidic plate and covering the fluidic network, the fluidic network being connected, at a first end, to an inlet orifice allowing entry of a liquid to be sprayed and, at a second end, to a first end of an outlet channel for the liquid to be sprayed, formed in the fluidic plate and extended by an electrospray nozzle in the shape of a pointed tip at which the second end of the outlet channel forms the electrospray outlet of the lab-on-a-chip, the cover plate having a pointed extension forming a roof for the channel part located in the electrospray nozzle.

According to one particular embodiment, the support plate has a pointed extension forming a floor for the channel part located in the electrospray nozzle. According to one variant of embodiment, the second end of the outlet channel, forming the electrospray outlet, is recessed relative to the pointed extensions forming the roof and floor.

The inlet orifice can be a hole formed in the cover plate or support plate.

The cover plate may be in silicon.

The support plate, on the fluidic plate side, may comprise a protective layer able to protect the remainder of the support plate during the formation of the fluidic network in the fluidic plate. The fluidic plate may be in silicon. In this case, according to one variant of embodiment, the fluidic plate, the protective layer, and the remainder of the support plate respectively derive from the thin layer, the buried oxide layer and the support of one same silicon-on-insulator substrate.

The cover plate can be electrically conductive.

### BRIEF DESCRIPTION OF THE DRAWINGS

The invention will be better understood and other advantages and aspects will become apparent on reading the following description given as a non-limiting example, accompanied by the appended drawings among which:

FIG. 1 is a diagram of a lab-on-a-chip according to the present invention,

FIG. 2 shows the COMOSS structure of an enzyme digestion reactor used in the lab-on-a-chip in FIG. 1,

FIG. 2A shows a detail of FIG. 2,

FIG. 3 shows the COMOSS structure of a pre-concentration reactor used in the lab-on-a-chip in FIG. 1,

FIG. 3A shows a detail of FIG. 3,

FIG. 4 shows the COMOSS structure of a chromatography reactor used in the lab-on-a-chip in FIG. 1,

FIG. 4A shows a detail of FIG. 4,

FIGS. 5A to 5D are cross-sectional views of a cover plate in progress of fabrication,

FIG. 5D′ is a perspective view of the cover plate in progress of fabrication,

FIGS. 5E to 5G are cross-sectional views of a support plate in progress of fabrication,

FIG. 5F′ is a perspective view of the support plate in progress of fabrication,

FIG. 5H is a cross-sectional view of the assembling of a support plate and a fluidic plate,

FIGS. 5I to 5K are cross-sectional views of the assembling of a support plate and fluidic plate, the fluidic plate being in progress of being machined,

FIGS. 5L and 5M are cross-sectional views of the assembling of the cover plate on the assembly consisting of the fluidic plate on the support plate,

FIGS. 5N and 5O are cross-section views illustrating the last fabrication steps of a lab-on-a-chip according to one embodiment of the present invention,

FIG. 6 is a partial, perspective view of a lab-on-a-chip according to a first variant of the invention,

FIG. 7 is a partial, perspective view of a lab-on-a-chip according to a second variant of the invention,

FIGS. 8A to 8E illustrate a variant of embodiment of a lab-on-a-chip according to the invention, using a SOI substrate.

### DETAILED DESCRIPTION OF PARTICULAR EMBODIMENTS

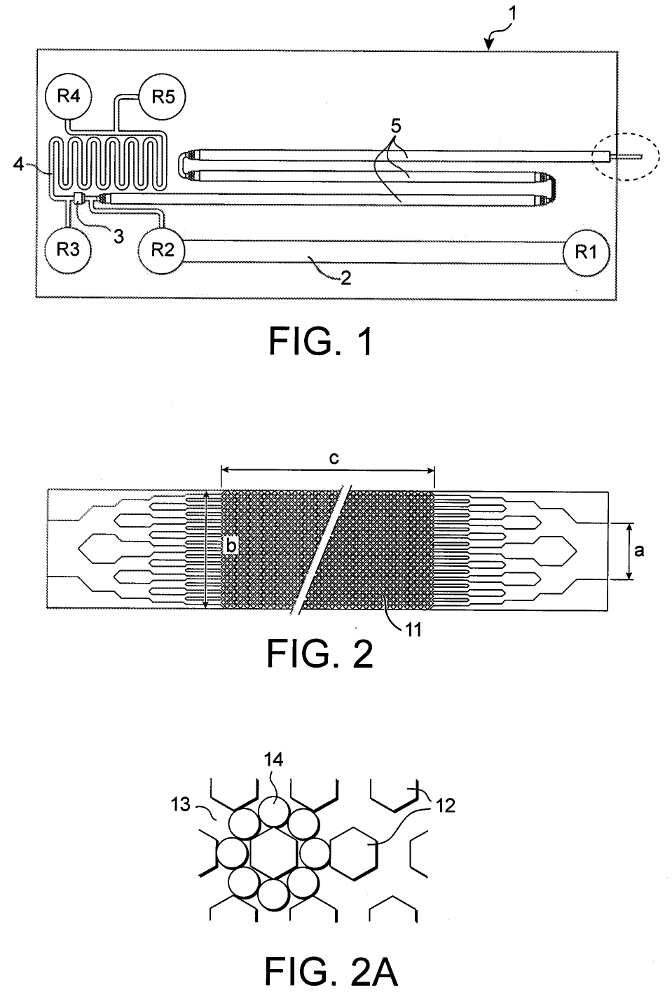

FIG. 1 is a diagram of a lab-on-a-chip 1 to which the present invention applies. This device may have a length of 18 mm and width of 5 mm.

The fluidic network

The fluidic network is first described which is intended to prepare a complex biological sample for identification of its protein content. This fluidic network consists of an assembly of reservoirs and channels, an enzyme digestion reactor, a pre-concentration reactor and a separation reactor by liquid electro-chromatography. The basic structure of all these reactors is a deep cavity provided with a large number of pillars of square or hexagonal section. This type of structure is known as a COMOSS (Collocated MOnolith Support Structure). In this respect, reference can be made to the article by Bing He et al: “Fabrication of nanocolumns for liquid chromatography”, Anal. Chem. 1998, 70, 3790-3797. For all reactors, benefit is drawn from the large surface/volume ratios developed by these COMOSS structures, these ratios increasing the <<meeting>> probabilities between molecules in the mobile phases (e.g. proteins for the enzyme digestion reactor) and those in the stationary phases (e.g. trypsin for the enzyme digestion reactor).

After complete pre-filling of the fluidic network with buffer, the biological sample (protein) is placed in reservoir RI and then pumped under electro-osmosis from reservoir R1 to reservoir R2 through the enzyme digestion reactor 2. Large volume reservoirs are arranged between the different reactors of the fluidic network so as to allow change of buffer between two consecutive steps of the protocol. Therefore R1 contains ammonium bicarbonate ([NH₄HCO₃]=25 mM; pH=7.8), R2, R3 and R4 contain a mixture of water/acetonitrile ACN/formic acid TFA (95%; 5%; 0.1%), whilst R5 contains a mixture of water/acetonitrile/formic acid (20%; 80%; 0.1%). The digest collected in reservoir R2 must be concentrated before separation. For this purpose, it is pumped under electro-osmosis towards reservoir R3 (bin). All the peptides resulting from enzymatic digestion are then <<captured>> by the small volume pre-concentration reactor 3, hence the concentration. An acetonitrile gradient, formed of a mixture of R4 buffer and R5 buffer in the structure 4 of <<serpentine>> type (2 cm in length), then selectively separates the peptides according to their affinity with the stationary phase (C18 for example) in the pre-concentration reactor 3. These are again <<captured>> by the chromatography column 5, denser than the pre-concentration reactor 3. Enriching the mixture with ACN again allows selective separation of these peptides from the chromatography column 5 and transfer thereof, separated, towards the outlet 6 of the chip 1 where the liquid is sprayed towards the inlet of a mass spectrometer, not shown.

A reactor having affinity with a given protein (not shown) can be used to capture this protein in a multi-protein mixture conveyed through this reactor. For this purpose, upstream of the above-described fluidic network, an assembly of reservoirs/affinity reactor/concentration reactor can be integrated operating along the same fluidic principles as previously described. The affinity reactor can be functionalized with antibodies and the elution buffer can consist of proteins that are concurrent (vis-a-vis the antibody) with the one it is desired to <<capture>> in the multi-protein complex.

The Upstream Affinity Reactor

Of COMOSS structure, it is intended for the specific capture of a protein, a family of proteins, or multi-protein complex in the complex biological sample. The tools used for this step may be antibodies, but may also be small molecules for example which have specific interaction with the desired protein(s).

The Enzyme Digestion Reactor

The COMOSS structure of the enzyme digestion reactor, shown FIG. 2, is formed from an array of pillars of 10 μm hexagonal section allowing a network of channels to be defined of around 5 μm. Its effective width a is constant (640 μm), but its actual width b measures 892 μm. The length c of the active part of the reactor is 15 mm. Its other geometric characteristics, to be read with reference to FIG. 2, are described in the following table:

| | Channel | Separating walls |
|---|---|---|
| Entity | width (μm) | (μm) |
| Connecting | 640 | 0 |
| channel | | |
| Stage 1 | 2 * 320 | 1 * 128 |
| Stage 2 | 4 * 160 | 3 * 64 |
| Stage 3 | 8 * 80 | 7 * 32 |
| Stage 4 | 16 * 40 | 15 * 16 |
| Stage 5 | 32 * 20 | 31 * 8 |
| Stage 6 | 64 * 10 | 63 * 4 |

This structure optionally allows the arrangement of silica <<beads>> or microspheres of a few micrometers (Microspheres by Bangs Laboratories distributed in France by Serotec for example) which are functionalized (e.g. Trypsin) to provide the reactor with its enzymatic properties or to increase such properties.

For example, the enzyme grafted on the pillars may be trypsin. The protocol followed is the one described in document FR-A-2 818 662.

FIG. 2A shows a detail of the reactor region referenced 11 in FIG. 2. The pillars 12 of hexagonal section can be seen in this figure defining the network of channels 13. Reference 14 designates the silica microspheres which may optionally be used.

The Pre-Concentration Reactor

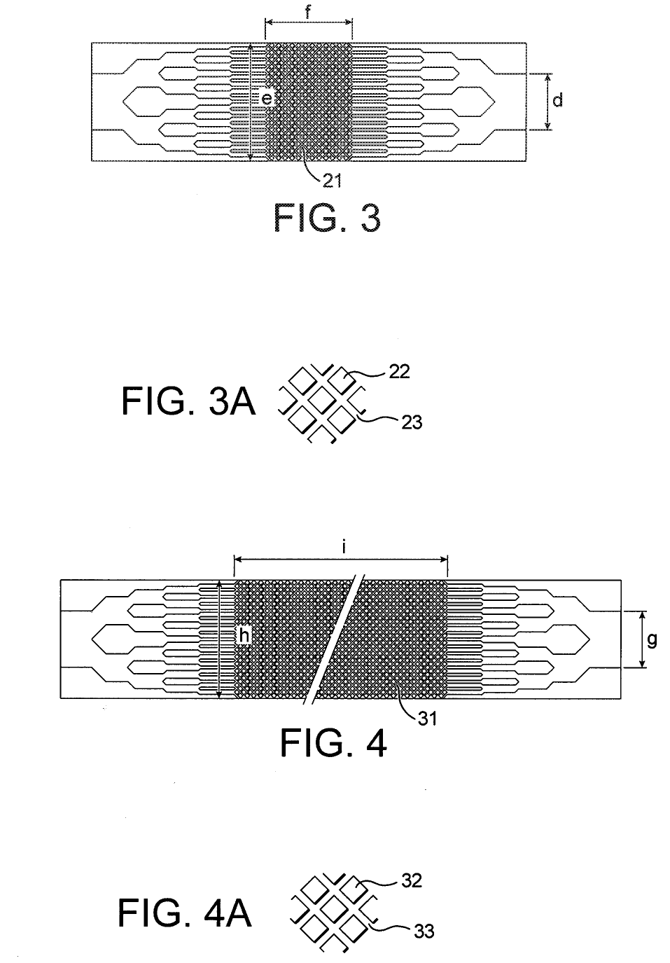

The COMOSS structure of the pre-concentration reactor, shown FIG. 3, is formed of an array of pillars of 10 μm square section defining a network of channels of around μm. Its effective width d is constant (160 μm), but its actual width e measures 310 μm. The length f of the active part of the reactor measures 170 μm. Its other geometric characteristics, to be read with reference to FIG. 3, are described in the following table:

| | Channel Width | Separating walls |
|---|---|---|
| Entity | (μm) | (μm) |
| Connecting | 160 | 0 |
| channel | | |
| Stage 1 | 2 * 80 | 1 * 80 |
| Stage 2 | 4 * 40 | 3 * 40 |
| Stage 3 | 8 * 20 | 7 * 20 |
| Stage 4 | 16 * 10 | 15 * 10 |

This structure allows optional organisation of silica beads which are functionalized to provide the reactor with its affinity properties or to increase such properties (C18 grafting for example).

FIG. 3A shows a detail of the region of the reactor referenced 21 in FIG. 3. The pillars 22 of square section can be seen, which allow defining of the network of channels 23.

The Separation Reactor by Liquid Electro-Chromatography

The COMOSS structure of the separation reactor, shown FIG. 4, is formed from an array of pillars of 10 μm square section allowing a network of channels to be defined of around 2 μm. Its effective width g is constant (160 μm), but its actual width h measures 310 μm. The length i of the active part of the reactor is 12 mm. Its other geometric characteristics, to be read with reference to 1a FIG. 4, are described in the following table:

| Entity | Channel width (μm) | Separating walls (μm) |
|---|---|---|
| Connecting channel | 160 | 0 |
| Stage 1 | 2 * 80 | 1 * 80 |
| Stage 2 | 4 * 40 | 3 * 40 |
| Stage 3 | 8 * 20 | 7 * 20 |
| Stage 4 | 16 * 10 | 15 * 10 |

To save space, the reactor can be made in three parts each having a length of 12 mm as shown FIG. 1.

This structure optionally allows the organizing of silica beads which are functionalized to provide the reactor with its affinity properties or to increase such properties (C18 grafting for example).

FIG. 4A shows a detail of the region of the reactor referenced 31 in FIG. 4. It shows the pillars 32 of square section, which allow defining of the network of channels 33.

### The Electrospray Source

One embodiment of the present invention will now be described in detail.

A preferred configuration is based on the use of hydrodynamics to set in movement the liquids to be sprayed by means of high pressure pumps, and not by electro-osmosis. This leads to the omission of some of the electrodes required for the device disclosed in document WO-A-2005/076 311. The electrode needed for placing the liquid to be sprayed at a given potential consists of the cover for example which is chosen to be in electrically conductive material. One variant consists of choosing an electrically insulating cover, fluidic plate and support plate. This may be obtained by thermal oxidation if these plates are in silicon. In this case, the electric potential can be imposed by means of a commercially available liquid junction arranged at the inlet to the device at its connection with the inlet capillary.

One advantageous embodiment of the present invention lies in the structuring and assembling of three silicon plates (support plate, fluidic plate and cover plate), their thinning by physicochemical polishing and DRIE etching (Dry Reactive Ion Etching). The assembling of the plates can advantageously be obtained by molecular bonding or wafer bonding.

The forming of the fluidic network in the fluidic plate will not be detailed in the remainder of the description. Reference for such forming can be made to document WO-A-2005/076 311. In the example of embodiment described below, the fluidic network is coplanar with the channel of the ESI source allowing coupling with no dead volume (no bend, no restricted section, . . . ). The fluidic network may chiefly consist of channels of square section and dimensions of 15 μm×15 μm.

The technology followed uses silicon plates 200 mm in diameter to form a plurality of devices. The dimensions of these plates are given by way of example, as are their thickness and their properties.

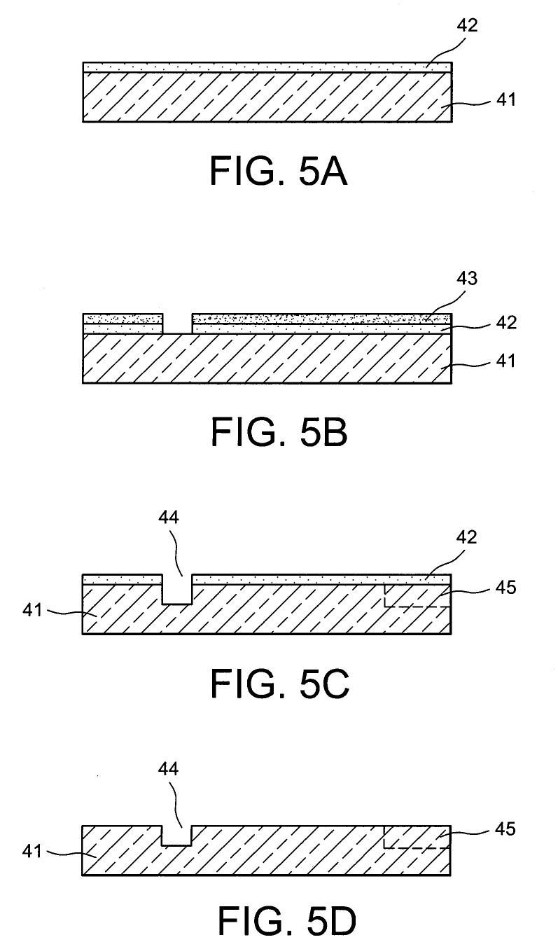

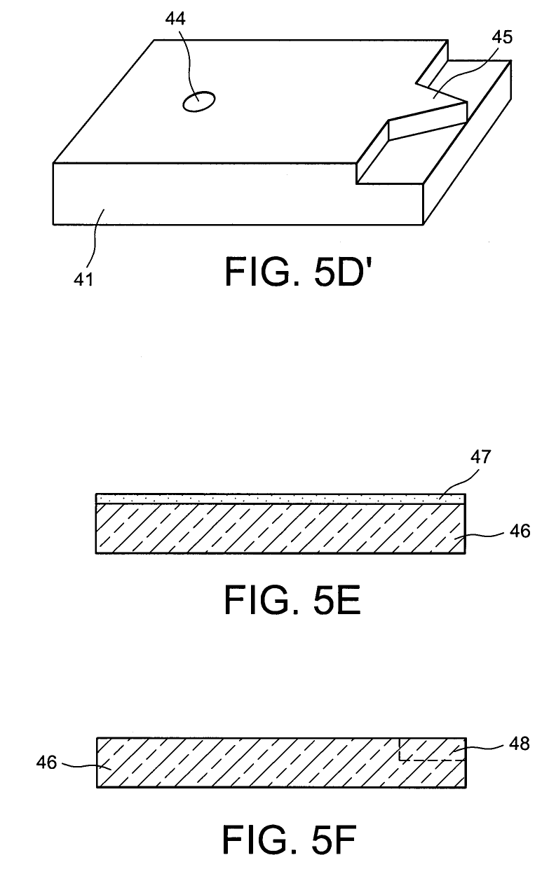

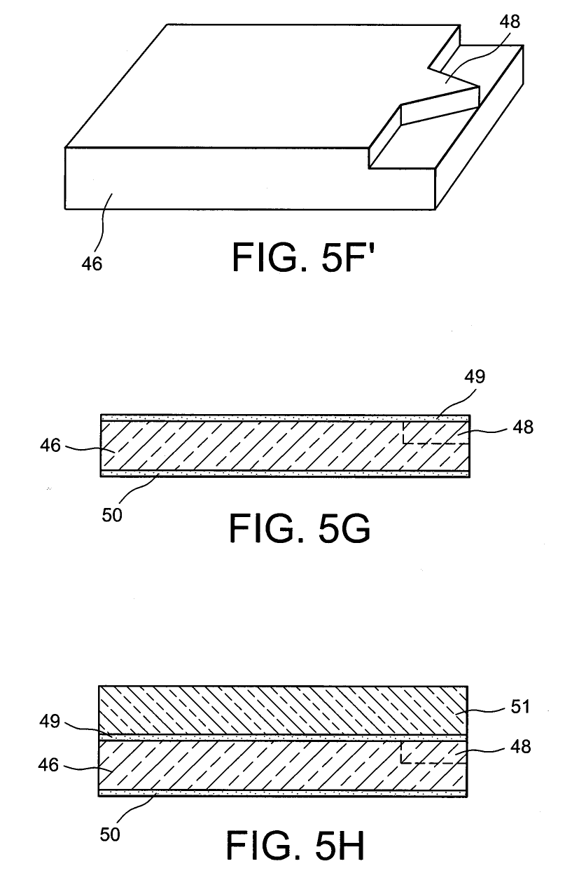

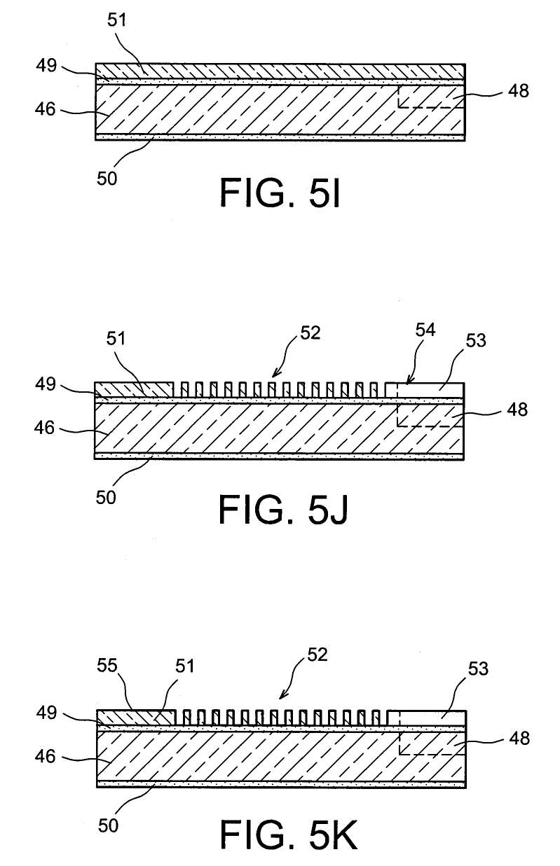

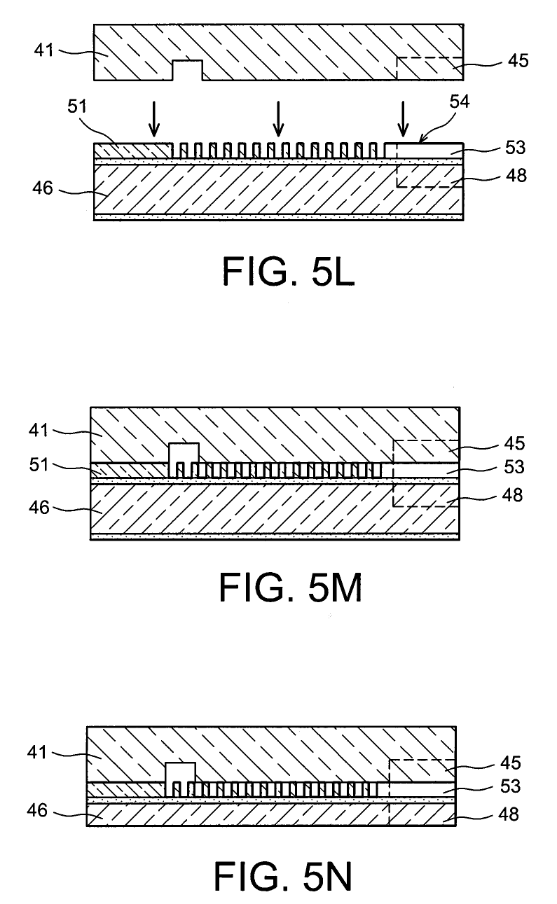

FIGS. 5A to 5D are cross-sectional views of a cover plate in the progress of being fabricated, the cross-section being taken along the longitudinal axis of the plate. FIG. 5D′ is a perspective view of the cover plate at this stage of fabrication.

FIG. 5A shows a fraction (corresponding to a cover of the device) of a silicon plate 41 with a diameter of 200 mm, polished on one face and having electrical conductivity of between 0.01 and 0.02 Ω·cm. The polished face of the plate 41 is coated with a silicon oxide layer 42 of thickness 2.5 μm formed by PECVD (Physical Enhanced Chemical Vapour Deposition). This oxide layer will act as etching mask.

The etching mask is then structured by photolithography. For this purpose, a layer of resin 43 is deposited which is then photo-lithographed (see FIG. 5B). Lithography defines a pattern in the resin layer 43 exposing the oxide layer 42. The resin is then removed. Next a blind hole 44, intended to form the inlet orifice of the device, is formed in the plate 41 by DRIE etching. The same mask and the same etching cause the defining and etching of the straight part of the cover plate which, in the upper part of the cover plate 41 and along its longitudinal axis, forms a pointed extension 45. The depth of etching is 170 μm for example.

The oxide mask is then removed. This (see FIG. 5D) gives a plate partly etched with a fluidic inlet orifice 44 and a pointed extension 45 which is part of the ESI source.

FIG. 5D′ is a perspective view corresponding to the cross-sectional view in FIG. 5D and providing a better illustration of the extension of pointed shape 45.

FIGS. 5E to 5G are cross-sectional views of a support plate in progress of fabrication, the cross-section being taken along the longitudinal axis of the plate. FIG. 5F′ is a perspective view of the support plate.

FIG. 5E shows a fraction (corresponding to a support of the device) of a silicon plate 46 of diameter 200 mm, polished on its two faces and 550 μm thick. One of the faces of the plate 46 is coated with a silicon oxide layer 47 of thickness 2.5 μm formed by PECVD. This oxide layer will be used as etching mask.

The etching mask is then structured by photolithography. For this purpose, a resin layer is deposited which is then photo-lithographed. Lithography, after removal of the resin and DRIE etching of the straight part of the support plate 46 along its longitudinal axis, defines an extension 48 in the shape of a pointed tip. This extension is better visible FIG. 5F′.

The plate 46 is then thermally oxidized to provide oxide layers 49 and 50 on each face of the plate 46. The oxide layer 49 is evidently also formed on the etched parts of the plate 46 which are located below the pointed extension 48 (see FIG. 5F′). The thickness of these oxide layers 49 and 50 may be 1.5 μm. These oxide layers will act as etch stop layers for a subsequent etching step of the fluidic plate (see FIG. 5G).

FIG. 5H is a cross-sectional view of the assembling of the support plate and fluidic plate (in fact the plate intended to form the fluidic plate), the cross-section being taken along the longitudinal axis of these plates. This figure shows a fraction of the assembled plates (corresponding to a device). A so-called fluidic plate 51 in silicon of diameter 200 mm and thickness 550 μm is bonded, via one of its faces which is polished, onto the support plate 46. Joining is made by molecular bonding, the joining being made on the side of the pointed extension 48.

The fluidic plate 51, attached onto the support plate 46, is then thinned by physicochemical polishing until the designed thickness is obtained (e.g. 15 μm). This is shown FIG. 5I.

The thinned fluidic plate is then structured. This step is shown FIG. 5J. For this purpose, a resin mask (thickness 1.5 μm) is deposited on the thinned fluidic plate 51 and photo-lithographed using an appropriate pattern. Next, in the fluidic plate 51, the fluidic network 52 and the channel 53 of the ESI source are simultaneously fabricated using DRIE etching. The oxide layer 49 of the support acts as etch stop layer. The same etching, in the straight part of the fluidic plate 51 and along its longitudinal axis, allows a pointed extension 54 to be obtained which can be superimposed over the pointed extension 48 of the support plate 46 for example, and also provides the outlet channel 53.

Next, a silicon oxide layer 55 is formed on the structured fluidic plate 51 (see FIG. 5K). The thickness of the oxide formed may range from 0.1 to a few μm.

FIGS. 5L and 5M illustrate the assembling of the cover plate on the fluidic plate. The cover plate 41 (see FIG. 5D) and the fluidic plate 51, which is already bonded to the support plate 46, are aligned one above the other as shown FIG. 5L. The pointed extension 45 of the cover plate 41 is then aligned with the extensions 54 of the fluidic plate and 48 of the support plate. The cover plate 41 is then attached onto the fluidic plate 51 by molecular bonding (see FIG. 5M). The pointed extensions 48, 54 and 45 are therefore superimposed.

The support plate 46 is then thinned by physicochemical polishing to release the pointed extension 48. This is shown FIG. 5N.

It is then the turn of the cover plate 41 to be thinned to obtain release of the pointed extension 45 and to gain access to the hole 44. This step can be performed using physicochemical polishing starting from the free face of the cover plate 41. DRIE etching can be used to obtain good finishing of the opening of the hole 44. FIG. 50 shows the result obtained.

The separation of the devices into individual chips can be obtained by cutting, cleaving or breaking.

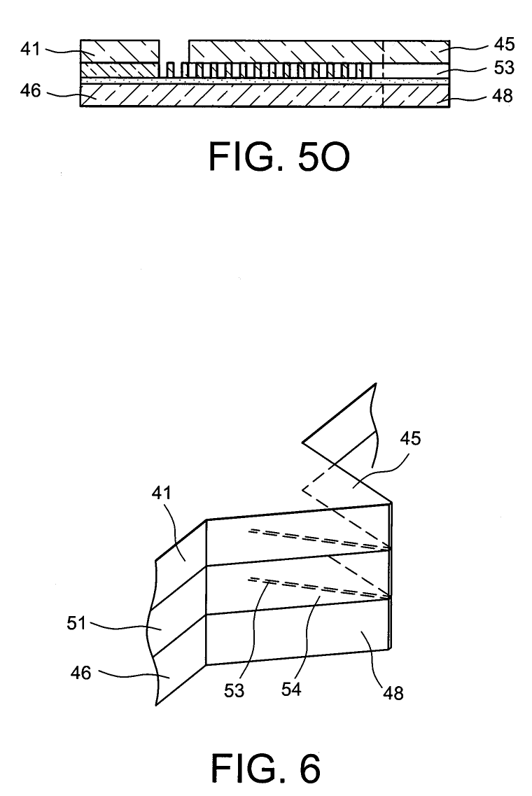

FIG. 6 is a partial, perspective view of a lab-on-a-chip according to the invention and obtained using the process just described. In this example of embodiment, the pointed extensions 45 of the cover plate 41, 54 of the fluidic plate 51, and 48 of the support plate 46 are of the same shape. The channel 53 of the ESI source, as far as the source outlet, is therefore provided with a floor consisting of extension 48 and with a roof consisting of extension 45.

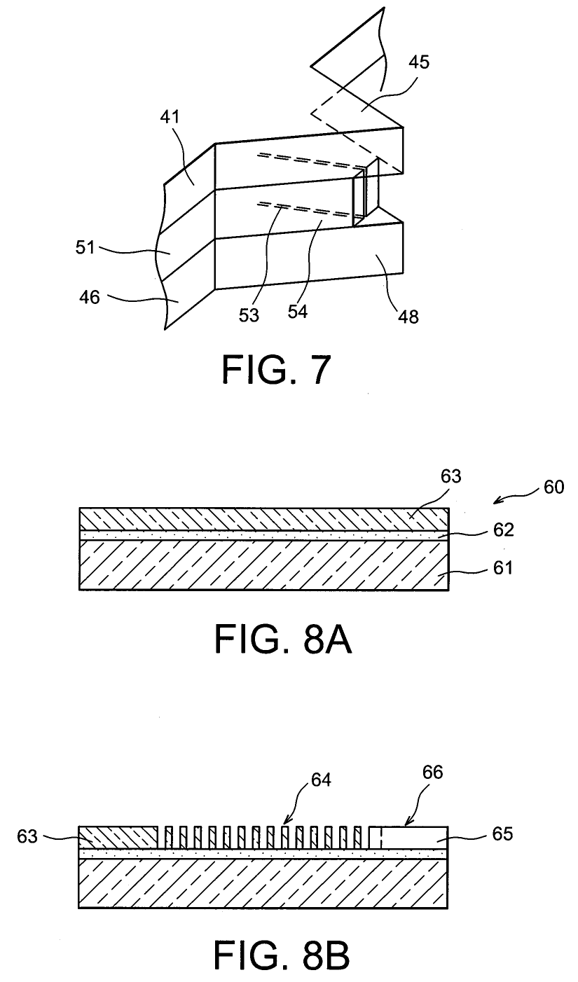

FIG. 7 is a partial, perspective view of another lab-on-a-chip according to the invention and obtained using the described method. In this example of embodiment, the tip of the pointed extension 54 of the fluidic plate 51 is truncated and is recessed relative to the tips of the pointed extensions 45 of the cover plate 41 and 48 of the support plate 46. This electrospray nozzle geometry can allow better stability of the Taylor cone.

Variants other than those shown FIGS. 6 and 7 are possible provided that the pointed extension of the cover plate continues to form a roof for the outlet channel. For example, the tip of the pointed extension of the support plate may be recessed relative to tip of the pointed extension of the fluidic plate, which may itself be recessed relative to the tip of the pointed extension of the cover plate.

Another embodiment of the present invention will now be described using a commercially available SOI substrate.

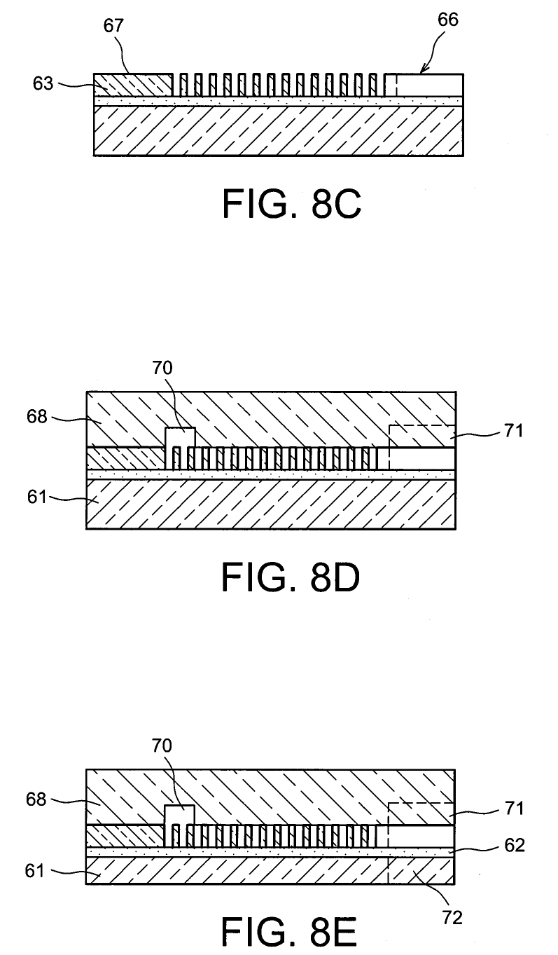

FIG. 8A is a cross-sectional view of a SOI substrate. The treatment of this substrate will be limited to the case of a single lab-on-a-chip for reasons of simplification. The SOI substrate 60 comprises a silicon support 61 successively supporting a buried silicon oxide layer 62 and a thin silicon layer 63. The diameter of the substrate 60 may be 200 mm. The thickness of the thin layer 63 may range from a few μm to a few tens μm. Its free face is polished. The thickness of the oxide layer may range from 0.1 μm to 3 μm. The thickness of the support 61 may be several hundred μm, e.g. 670 μm.

The top silicon layer 63 will be used as fluidic plate. FIG. 8B shows the structuring step of the fluidic plate. A resin mask (e.g. thickness of 1.5 μm) is deposited on the thin layer 63 and photo-lithographed according to the desired fluidic network pattern. Then, simultaneously and in the thin layer 63, the fluidic network 64 and the channel 65 of the ESI source are fabricated using DRIE etching. The buried oxide layer 62 acts as etch stop layer. The same etching allows a pointed extension 66 to be obtained in the straight part of the thin layer 63 and along its longitudinal axis.

Next, a silicon oxide layer 67 is formed on the structured thin layer 63 (see figure 8C). The thickness of the formed oxide may range from 0.1 to a few μm.

The cover plate is fabricated as in the preceding embodiment (see FIGS. 5A to 5D). It is then bonded onto the element shown FIG. 8C, by covering the fluidic network. This is illustrated FIG. 8D in which the structured cover plate is referenced 68. The cover plate 68 comprises the blind hole 70, intended to form the inlet orifice of the device, and the pointed extension 71. Next, a SiO₂ layer is deposited on the lower face of the support.

Photo-lithography is then performed starting from the lower face of the support plate. The oxide layer deposited on the lower face of the support is etched to act as mask, and the resin layer used for this photo-lithography is removed. DRIE etching is then conducted on the silicon of the support plate 61 to define the lower tip of the ESI source. The support plate 61 is then thinned by physicochemical polishing. FIG. 8E illustrates the result obtained. It shows the pointed extension 72 of the support plate 61. The oxide layer 62 is then etched at the ESI source to give the lower face of the source its final appearance.

The cover plate 68 is then thinned to obtain release of the pointed extension 71 and to gain access to the hole 70. This step can be conducted by physicochemical polishing starting from the free face of the cover plate 68, optionally followed by DRIE etching for finishing. The device obtained is then similar to the one illustrated FIG. 50.

The use of a SOI substrate provides the advantage that the support and fluidic plates are delivered bonded. Full plate bonding with no patterns guarantees better bonding yield. Another advantage is that the pair of steps, lithography/DRIE etching, which is the most difficult to implement for etching of the fluidic network, is conducted at the start of fabrication. This makes it possible to discard faulty plates as soon as possible and hence to increase final yield. The use of a SOI substrate also entails one less thinning operation by DRIE etching.

1. Lab-on-a-chip comprising a support plate, at least one fluidic network formed in a so-called fluidic plate bonded onto the support plate, and a so-called cover plate bonded onto the fluidic plate and covering the fluidic network, the fluidic network being connected, at a first end, to an inlet orifice allowing entry of a liquid to be sprayed and, at a second end, to a first end of an outlet channel for the liquid to be sprayed, formed in the fluidic plate which is extended by an electrospray nozzle of pointed shape at which the second end of the outlet channel forms the electrospray outlet of the lab-on-a-chip, characterized in that the cover plate has a pointed extension forming a roof for that part of the channel located in the electrospray nozzle.

2. Lab-on-a-chip according to claim 1, wherein the support plate has a pointed extension forming a floor for that part of the channel located in the electrospray nozzle.

3. Lab-on-a-chip according to claim 2, wherein the second end of the outlet channel, forming the electrospray outlet, is recessed relative to the pointed extensions forming a roof and floor.

4. Lab-on-a-chip according to claim 1, wherein said inlet orifice is a hole formed in the cover plate or support plate.

5. Lab-on-a-chip according to claim 1, wherein the cover plate is in silicon.

6. Lab-on-a-chip according to claim 1, wherein the support plate, on the fluidic plate side, comprises a protective layer able to protect the remainder of the support plate during formation of the fluidic network in the fluidic plate.

7. Lab-on-a-chip according to claim 1, wherein the fluidic plate is in silicon.

8. Lab-on-a-chip according to claim 6, wherein the fluidic plate, the protective layer and the remainder of the support plate respectively derive from the thin layer, the buried oxide layer and the support of one same silicon-on-insulator substrate.

9. Lab-on-a-chip according to claim 1, wherein the cover plate is electrically conductive.

10. Lab-on-a-chip according to claim 7, wherein the fluidic plate, the protective layer and the remainder of the support plate respectively derive from the thin layer, the buried oxide layer and the support of one same silicon-on-insulator substrate.
